(19) United States
(12) Patent Application Publication
(10) Pub. No.: US 2021/0261599 A1
(43) Pub. Date: Aug. 26, 2021

(54) Process for the Preparation of Obeticholic Acid and Intermediates Used In the Process Thereof

(71) Applicant: Solara Active Pharma Sciences Limited, Maharashtra (IN)

(72) Inventors: Anil Ganpatrao Holkar, Karnataka (IN); Vellaichamy Ganesan, Tamil Nadu (IN); Dhanapal Ramu, Tamil Nadu (IN); Manjunatha Bhat, Karnataka (IN); Prasad N Patgar, Karnataka (IN); Loganathan Mani, Tamil Nadu (IN); Sudarsanam Yeshaiah, Andhra Pradesh (IN); Swaminathan Srinivasan, Tamil Nadu (IN); Karabasanagouda Timmanagouda, Karnataka (IN)

(21) Appl. No.: 17/261,647

(22) PCT Filed: Mar. 15, 2019
(86) PCT No.: PCT/IN2019/050209

§ 371 Date: Jan. 20, 2021

(30) Foreign Application Priority Data
Aug. 24, 2018 (IN) 201841031853

(51) Int. Cl. C07J 9/00; C07J 75/00

(52) U.S. Cl. CPC C07J 9/005; C07B 2200/13; C07J 75/00

(57) ABSTRACT

The present invention relates to an improved process for the preparation of Obeticholic acid and intermediates used in the process thereof. The invention also relates to a solid form of tertiary butylamine salt of Obeticholic acid and tertiary butylamine salt of Obeticholic acid in the solvate form.

### RELATED PATENT APPLICATION(S)

This application claims the priority to and benefit of Indian Patent Application No. 201841031853 filed on Aug. 24, 2018; the disclosures of which are incorporated herein by reference.

### FIELD OF THE INVENTION

The present invention relates to an improved process for the preparation of Obeticholic acid and intermediates used in the process thereof. The invention also relates to a solid form of tertiary butylamine salt of Obeticholic acid used during the preparation of Obeticholic acid.

### BACKGROUND OF THE INVENTION

Obetichloic acid is chemically known as 3-alpha, 7-alpha-dihydroxy-6-alpha-ethyl-5-beta-cholan-24-oic acid having the Formula-I as mentioned below.

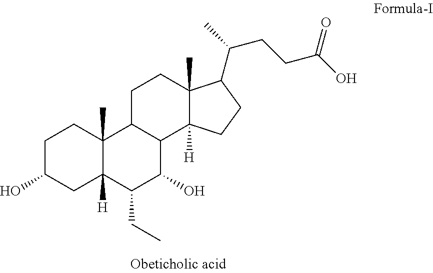 [SMILES: CC[C@H]1[C@@H](O)C2C(CC[C@]3(C)C([C@H](C)CCC(=O)O)CC[C@@H]23)[C@@]2(C)CC[C@@H](O)C[C@@H]12]

Obeticholic acid is a semi-synthetic bile acid analogue used for the treatment of primary bilary cholangitis in combination with ursodeoxycholic acid and is marketed in US as Ocaliva.

The compound Obeticholic acid or its pharmaceutically acceptable salts were first disclosed in the U.S. Pat. No. 7,138,390 assigned to Intercept Pharmaceuticals. The U.S. Pat. No. 8,969,330 discloses process for the preparation of Obeticholic acid as mentioned below:

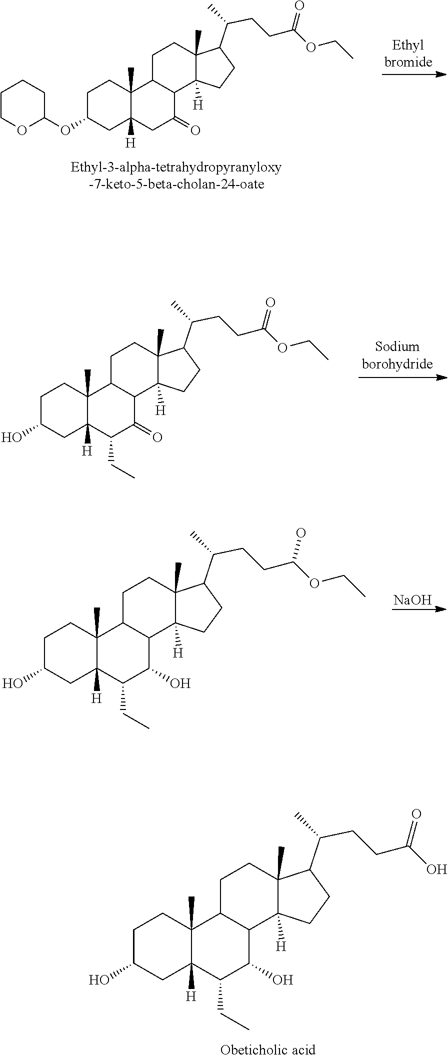

This process for the preparation of Obeticholic acid involves the alkylation of ethyl-3-alpha-tetrahydropyranyloxy-7-keto-5-beta-cholan-24-oate with ethyl bromide in the presence of n-butyl lithium and hexamethylene phosphonamide in a suitable solvent, followed by the reduction of the keto function in position-7 and de-protection of the acid function in position-24. All the stages of this process requires chromatographic separation that makes the process difficult at commercial scale.

The U.S. Pat. No. 7,812,011 discloses the preparation of Obeticholic acid as mentioned below:

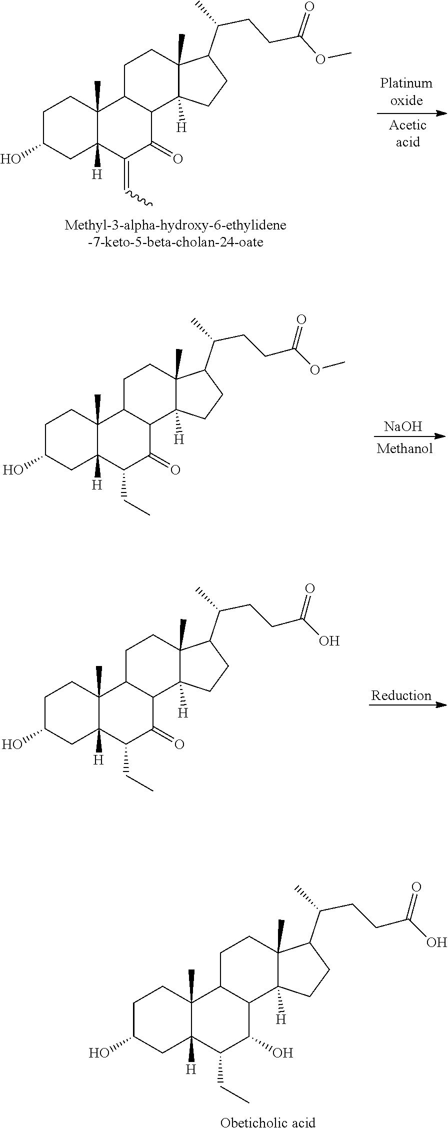

This process for the preparation of Obeticholic acid involves the reduction of methyl-3-alpha-hydroxy-6-ethylidene-7-keto-5-beta-cholan-24-oate in the presence of platinum oxide in acetic acid; followed by de-protection of the acid function in position-24 and reduction of the keto function in position-7. All the stages of this process requires chromatographic separation that makes the process difficult at commercial scale.

The U.S. Pat. No. 7,994,352 discloses an improved preparation of Obeticholic acid as mentioned below:

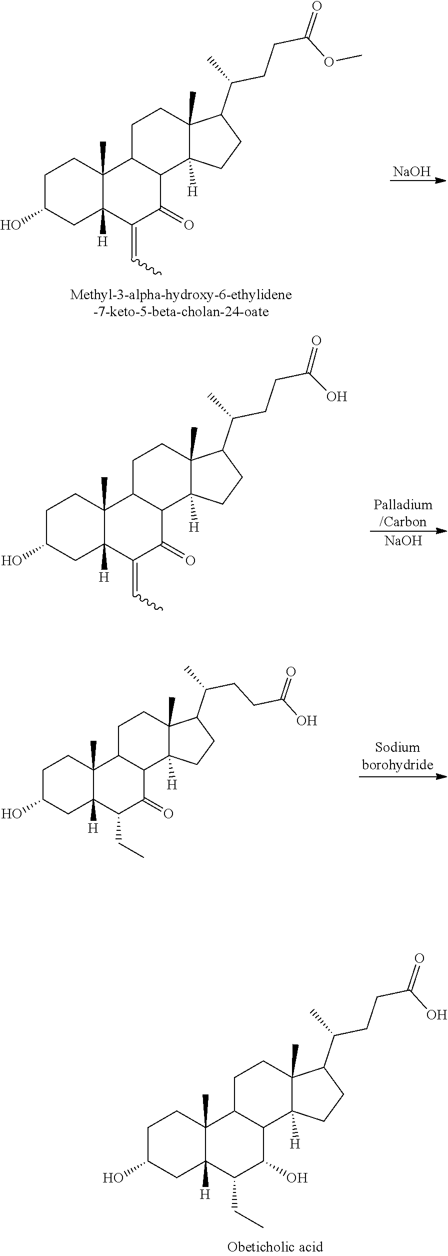

This process for the preparation of Obeticholic acid involves the deprotection of the acid function in position-24 of methyl-3-alpha-hydroxy-6-ethylidene-7-keto-5-beta-cholan-24-oate, followed by reduction of 3-alpha-hydroxy-6-ethylidene-7-keto-5-beta-cholan-24-oic acid in the presence of palladium carbon; and reduction of the keto function in position-7. The amorphous form of the Obeticholic acid was prepared by precipitating the Obeticholic acid from an aqueous ammonia solution by the addition of phosphoric acid.

There is a great need for higher purity in active pharmaceutical ingredients (API), since that would advantageously display improved pharmaceutical effectiveness than API with less purity. 6-Beta-ethylchenodeoxycholic acid and chenodeoxycholic acid are some of impurities of Obeticholic acid known in the state of art.

Separation by chromatographic purification methods as disclosed in the above cited patents is difficult since the physiochemical properties including partition coefficient and total surface polarity of the Obeticholic acid and its impurities are similar.

Crystallization is another conventional technique that has long been used in the purification of pharmaceutical substances. The purity of the final crystal product can be controlled either by proper selection of the process variables such as the solvent type, the degree of local and average super saturation, degree of mixing, crystallizer geometry, and seeding policy, or by implementing external control.

The U.S. Pat. No. 7,994,352 discloses the purification of Obeticholic acid involving the step of crystallizing in dichloromethane. The U.S. Pat. No. 9,238,673 disclose different solvents such as acetonitrile, heptane, nitromethane and butyl acetate for the purification of Obeticholic acid.

Salt formation is another simple and efficient approach that alter product's physiochemical properties that help in the purification of pharmaceutical substances.

The U.S. Pat. No. 7,138,390 teaches the preparation of amine salts of Obeticholic acid reacting Obeticholic acid with amine by conventional techniques and the use of the prepared salts in the preparation of Obeticholic acid free base.

The International Publication WO2017137931 having priority of Feb. 10, 2016 discloses the preparation of (S)-alpha-methyl-benzylamine and diethylamine salt of Obeticholic acid and the use of the same for the preparation of pure Obeticholic acid.

The EP Publication No. 3305799 having priority of Oct. 7, 2016, discloses the preparation of L-Arginine, L-Lysine, ammonia, tris(hydroxymethyl)aminomethane and potassium salts of Obeticholic acid.

The International Publication WO2018165269 having priority of Mar. 8, 2017 discloses Obeticholic acid monoammonium salt and cocrystals of Obeticholic acid.

Besides the availability of different methods for the preparation of Obeticholic acid in state of the art, there is a need for an improved process for the preparation of Obeticholic acid that is simple and economically significant at a large scale.

### OBJECT OF THE INVENTION

The object of the invention is to provide an improved process for the preparation of Obeticholic acid.

Another object of the invention is provide a process for the preparation of Obeticholic acid in amorphous form.

Another object of the invention is to provide a solid form of tertiary butylamine salt of Obeticholic acid and the process for preparation thereof.

Yet another object of the invention is to provide the tertiary butylamine salt of Obeticholic acid in the form of solvate and the process for preparation thereof.

### SUMMARY OF THE INVENTION

Accordingly, there is provided an improved process for the preparation of Obeticholic acid, amorphous form of Obeticholic acid, tert-butylamine salt of Obeticholic acid, and solvate of tert-butylamine salt of Obeticholic acid.

The main aspect of the invention is to provide an improved process for the preparation of

Obeticholic acid of formula-I having chenodeoxycholic acid impurity less than 0.5% w/w,

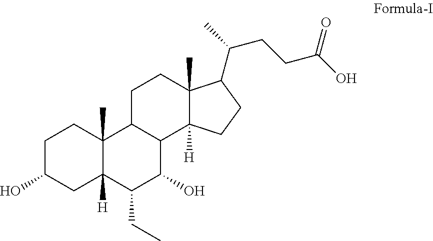 [SMILES: CC[C@H]1[C@@H](O)C2C(CC[C@]3(C)C([C@H](C)CCC(=O)O)CC[C@@H]23)[C@@]2(C)CC[C@@H](O)C[C@@H]12]

said process comprising the steps of:
(i) converting the compound of formula-III or its salt thereof to a compound of formula-II or its salt thereof

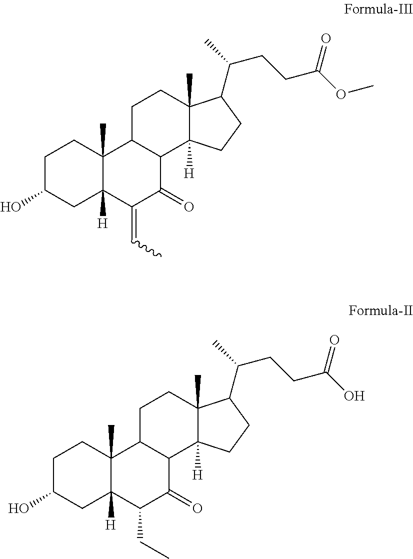

in the presence of palladium carbon in aqueous alkaline medium;
(ii) reducing the compound of formula-II or its salt thereof in the presence of sodium borohydride in an aqueous alkaline medium to obtain Obeticholic acid of formula-I;
(iii) treating Obeticholic acid of formula-I with tertiary butylamine to obtain a tertiary butylamine salt of Obeticholic acid; and
(iv) converting the tertiary butylamine salt of Obeticholic acid to Obeticholic acid of formula-I.

In some embodiment of the invention, the Obeticholic acid of formula-I obtained in the above described process is the amorphous form of Obeticholic acid.

Another aspect of the invention is to provide a process for the preparation of obeticholic acid comprising the steps of:
(i) purifying the compound of formula-II or its salt thereof,

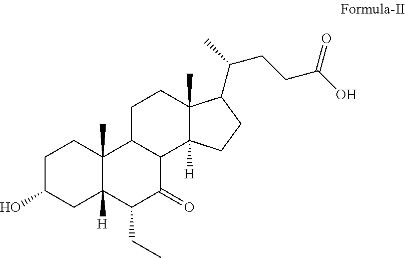 [SMILES: CC[C@H]1C(=O)C2C(CC[C@]3(C)C([C@H](C)CCC(=O)O)CC[C@@H]23)[C@@]2(C)CC[C@@H](O)C[C@@H]12]

comprising the steps of:
(a) dissolving the compound of formula-II or its salt thereof in a ketone solvent; and
(b) adding water to the solution obtained in step (a);
(c) isolating the purified form of the compound of formula-II or its salt thereof from the solution obtained in step (b);

(ii) reducing the purified form of the compound of formula-II or its salt thereof obtained in step
(i) in the presence of sodium borohydride in an aqueous alkaline medium to obtain Obeticholic acid of formula-I or its salt thereof; and

(iii) optionally purifying the Obeticholic acid of formula-I or its salt thereof.

Another aspect of the invention is to provide a solid form of tertiary butylamine salt of Obeticholic acid. In some embodiment of the invention, solid form of tertiary butylamine salt of Obeticholic acid is characterized by X-ray diffraction spectrum having peaks expressed as 2θ values at about 9.8, 11.6, 13.8, 14.6, 16.9, 17.9 and 18.51±0.2 degrees.

Still another aspect of the invention is to provide a process for the preparation of tertiary butylamine salt of Obeticholic acid comprising the steps of:
(a) providing a solution of Obeticholic acid or its salt thereof in one or more suitable solvent;
(b) adding tertiary butylamine to the solution of step (a);
(c) isolating tertiary butylamine salt of Obeticholic acid in solid form obtained in step (b);
(d) optionally, purifying the isolated tertiary butylamine salt of Obeticholic acid at a suitable temperature.

Yet another aspect of the invention is to provide a process for preparation of amorphous form of Obeticholic acid comprising the step of converting tertiary butylamine salt of Obeticholic acid to amorphous form of Obeticholic acid. In some embodiment of the invention, the said process for the preparation of amorphous form of Obeticholic acid comprises the steps of:
(a) adding tertiary butylamine salt of Obeticholic acid to mixture of a water and water immiscible solvent;
(b) adjusting the pH of the biphasic mixture obtained in step (a) between 2-5;
(c) separating the organic layer from the biphasic mixture obtained in step (b)
(d) adding water to the separated organic layer obtained in step (c);
(e) adding alkali carbonate or bicarbonate solution to the biphasic mixture obtained in step (d) to adjust the pH between 9-13;
(f) separating the aqueous layer from the biphasic mixture obtained in step (e);
(g) adjusting the pH between 3-5;
(h) optionally, stirring till the solid formation; and
(i) isolating the amorphous form of Obeticholic acid.

Another aspect of the present invention is to provide a tertiary butylamine salt of Obeticholic acid in the form of solvate. In some embodiment of the invention, the solvate form of tertiary butylamine salt of Obeticholic acid is alcohol selected from ethanol, methanol, propanol, isopropanol, butanol or pentanol or ketone selected from acetone or isobutyl ketone. In some embodiment of the invention, the solvate form of tertiary butylamine salt of Obeticholic acid is methanolate.

In some embodiment of the invention, the solvate form of tertiary butylamine salt of Obeticholic acid is methanol solvate. In some embodiment of the invention, the said methanol solvate of tertiary butylamine salt of Obeticholic acid is characterized by X-ray diffraction spectrum having peaks expressed as 2θ values at about 9.1, 10.9, 14.8, and 18.3±0.2 degrees.

Still another aspect of the invention is to provide a process for the preparation of amine salt of Obeticholic acid in form of solvate, comprising the steps of:
(a) dissolving a solid form of amine salt of Obeticholic acid in a suitable solvent selected from water, alcohol, ketone or mixture thereof; and
(b) isolating amine salt of Obeticholic acid in the form of solvate in solid form from the solution obtained in step (a).

In some embodiment of the invention, in the above described process for the preparation of amine salt of Obeticholic acid in form of solvate, the amine salt of Obeticholic acid is tertiary butylamine salt of Obeticholic acid.

### BRIEF DESCRIPTION OF THE FIGURES

FIG. 1: The PXRD pattern of tertiary butylamine salt of Obeticholic acid

FIG. 2: The PXRD pattern of amorphous form of Obeticholic acid of formula-I

FIG. 3: The PXRD pattern of tertiary butylamine salt of Obeticholic acid methanol solvate

### DETAILED DESCRIPTION OF THE INVENTION

The inventors of the present invention have developed an improved process for the preparation of Obeticholic acid with enhanced purity and feasible for large scale production.

The term “solvate” as used herein refers to crystalline solid adducts containing either stoichiometric or nonstoichiometric amounts of a solvent incorporated within the crystal structure.

One of the main aspect of the invention is to provide an improved process for the preparation of Obeticholic acid of formula-I having chenodeoxycholic acid impurity less than 0.5% w/w,

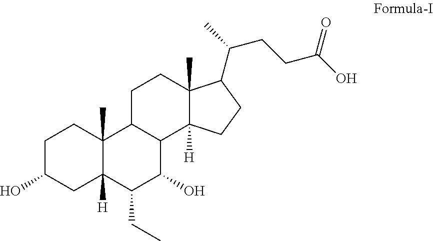 [SMILES: CC[C@H]1[C@@H](O)C2C(CC[C@]3(C)C([C@H](C)CCC(=O)O)CC[C@@H]23)[C@@]2(C)CC[C@@H](O)C[C@@H]12]

said process comprising the steps of:
(i) converting the compound of formula-III or its salt thereof to a compound of formula-II or its salt thereof.

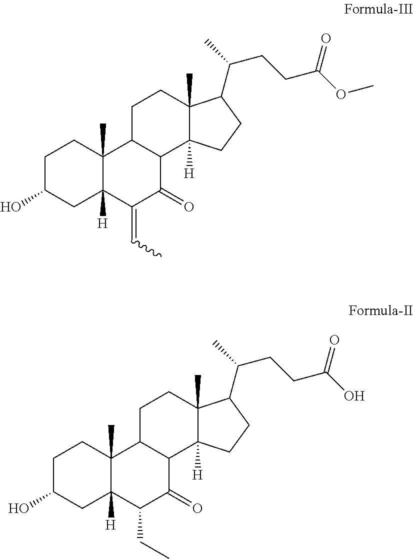

in the presence of palladium carbon in aqueous alkaline medium;
(ii) reducing the compound of formula II or its salt thereof in the presence of sodium borohydride in an aqueous alkaline medium to obtain Obeticholic acid of formula-I;
(iii) treating Obeticholic acid of formula-I thereof with tertiary butylamine to obtain a tertiary butylamine salt of Obeticholic acid; and
(iv) converting tertiary butylamine salt of Obeticholic acid to Obeticholic acid of formula-I.

In some embodiment of the invention, the Obeticholic acid of formula-I obtained in the above described process is the amorphous form of Obeticholic acid. In some embodiment of the invention there is provided a process for the preparation of amorphous form of Obeticholic acid having chenodeoxycholic acid impurity less than 0.5% w/w

In some embodiment of the invention, the conversion of the compound of formula-III to the compound of formula-II according to step (i) of the above described process is carried out in presence of alcoholic solvent, preferably methanol.

The conversion of the compound of formula-III to the compound of formula-II according to step (i) of the present process includes the hydrogenation reaction followed by hydrolysis of the methyl ester at the C-24 position and epimerization of the 6-beta-ethyl-group. In some embodiment of the invention, the hydrogenation reaction is preferably carried out in an alkaline aqueous medium along with organic solvent environment with pressure between 1 and 6 atmospheres.

In some embodiment of the invention, the conversion of the compound of formula-III to the compound of formula-II according to step (i) of the present process is carried out at temperatures between 80° C. and 105° C.

Chenodeoxycholic acid is major impurity present in Obeticholic acid that decreases the potency of Obeticholic acid. The removal of chenodeoxycholic acid from Obeticholic acid is difficult and repeated recrystallization in organic solvents decreases the yield of the final product. Hence, the formation of t-butylamine salt of Obeticholic acid, its purification and use in the preparation of Obeticholic acid free base effectively removes the chenodeoxycholic acid impurity below 2% w/w in the final API in amorphous form.

Another embodiment of the invention is to provide a process for the preparation of Obeticholic acid comprising the steps of:
(i) purifying the compound of formula-II or its salt thereof

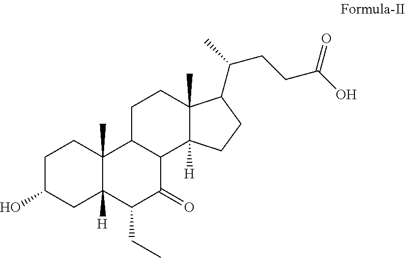 [SMILES: CC[C@H]1C(=O)C2C(CC[C@]3(C)C([C@H](C)CCC(=O)O)CC[C@@H]23)[C@@]2(C)CC[C@@H](O)C[C@@H]12]

comprising the steps of:
(a) dissolving the compound of formula-II or its salt thereof in a ketone solvent; and
(b) adding water to the solution obtained in step (a);
(c) isolating the purified form of the compound of formula-II or its salt thereof;
(ii) reducing the purified form of the compound of formula-II or its salt thereof obtained in step (i) in the presence of sodium borohydride in an aqueous alkaline medium to obtain obeticholic acid of formula-I or its salt thereof; and
(iii) optionally purifying the Obeticholic acid of formula-I or its salt thereof.

The ketone solvent according to step (a) include, but not limited to acetone, methyl isobutyl ketone or the like, preferably acetone or its mixture thereof.

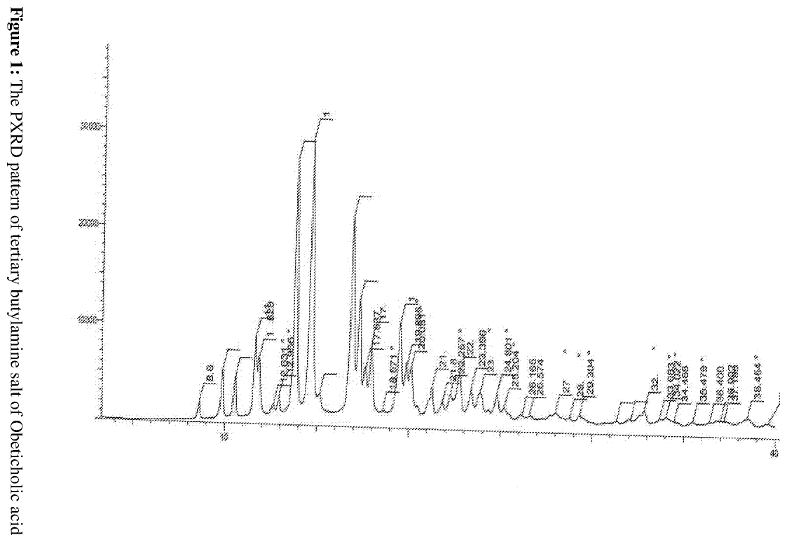

Another aspect of the invention is to provide a solid form of tertiary butylamine salt of Obeticholic acid. The PXRD pattern of the solid form of tertiary butylamine salt of Obeticholic acid is substantially illustrated in FIG. 1. The solid form of tertiary butylamine salt of Obeticholic acid of the present invention is characterized by X-ray diffraction spectrum having peaks expressed as 2θ values at about 9.8, 11.6, 13.8, 14.6, 16.9, 17.9 and 18.51°±0.2 degrees.

Another aspect of the invention is to provide a process for the preparation of tertiary butylamine salt of Obeticholic acid comprising the steps of:
(a) providing a solution of Obeticholic acid or its salt thereof in one or more suitable solvent;
(b) adding tertiary butylamine to the solution of step (a);
(c) isolating tertiary butylamine salt of Obeticholic acid in solid form obtained in step (b); and
(d) optionally, purifying the isolated tertiary butylamine salt of Obeticholic acid at a suitable temperature.

In some embodiment of the invention, the suitable solvent according to step (a) include, but not limited to alcohols such as methanol, isopropanol or the like; ketones such as acetone, methyl isobutyl ketone or the like; ether such as diethyl ether, tetrahydrofuran or the like; esters such as ethyl acetate, n-butyl acetate or the like; water or mixture thereof.

In some embodiment of the invention, tertiary butylamine in step (b) is added directly to the solution of Obeticholic acid in suitable solvent(s); or a solution of tertiary butylamine in suitable solvent(s) may be added to the solution of Obeticholic acid in suitable solvent(s). In some embodiment, the reverse addition may carried wherein the solution of Obeticholic acid in suitable solvent is added to tertiary butylamine in suitable solvent(s).

In some embodiment of the invention, the step (c) of the process is performed after formation of tertiary butylamine salt of Obeticholic acid. The formation of the tertiary butylamine salt of Obeticholic acid may be formed after addition of the tertiary butylamine; or after stirring the mixture containing tertiary butylamine and Obeticholic acid at a temperature between about 0° C. to 80° C. for 5 minutes to about 2 hours.

In some embodiment of the invention, the isolation, according to the step (c) of the process, of the tertiary butylamine salt of Obeticholic acid is carried out by any methods known in the state of art. Preferably after the formation tertiary butylamine salt of Obeticholic acid, the reaction mixture is cooled and the precipitated solid is isolated by filtration.

Preferably the purification of the isolated tertiary butylamine salt of obeticholic acid is carried out with one or more organic solvent by conventional methods known in the state of art. Said organic solvent include, but not limited to alcohols such as methanol, isopropanol or the like; ketones such as acetone, methyl isobutyl ketone or the like; ether such as diethyl ether, tetrahydrofuran or the like; esters such as ethyl acetate, n-butyl acetate or the like.

Another aspect of the present invention is to provide a tertiary butylamine salt of Obeticholic acid in the form of solvate. In some embodiment of the invention, the solvate form of tertiary butylamine salt of Obeticholic acid is alcohol selected from ethanol, methanol, propanol, isopropanol, butanol or pentanol or ketone selected from acetone or isobutyl ketone. In some embodiment of the invention, the solvate form of tertiary butylamine salt of Obeticholic acid is methanolate.

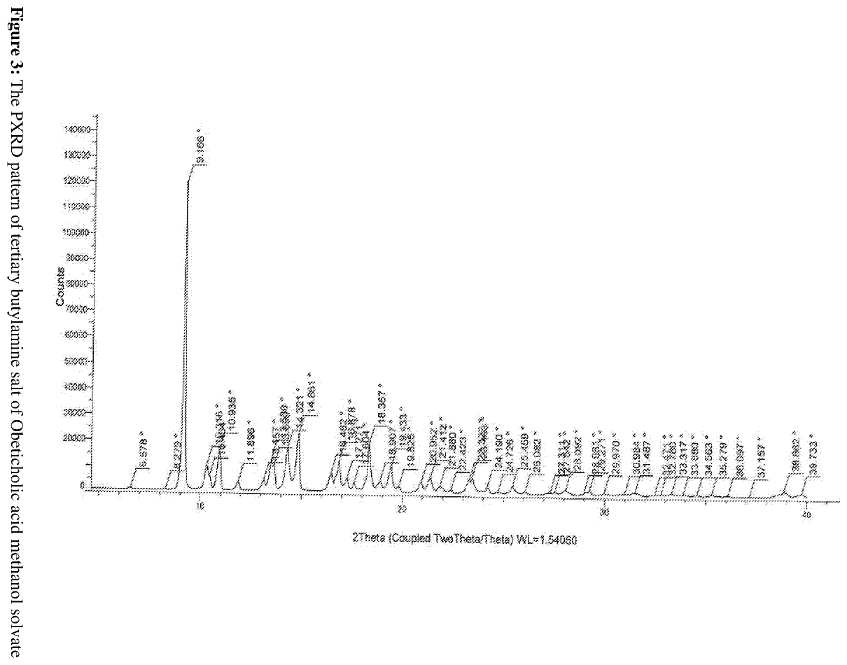

In some embodiment of the invention, the solvate form of tertiary butylamine salt of Obeticholic acid is methanol solvate. In some embodiment of the invention, the said methanol solvate of tertiary butylamine salt of Obeticholic acid is characterized by X-ray diffraction spectrum having peaks expressed as 20 values at about 9.1, 10.9, 14.8, and 18.3±0.2 degrees and illustrated in FIG. 3.

Yet another aspect of the invention is to provide a process for the preparation of amine salt of obeticholic acid in the form of solvate, comprising the steps of:
(a) dissolving a solid form of amine salt of Obeticholic acid in a suitable solvent or its mixtures thereof selected from the group comprising of water, alcohol, ketone or mixture thereof; and
(b) isolating amine salt of Obeticholic acid in the form of solvate in solid form from the solution obtained in step (a).

In some embodiment of the invention, in the above described process for the preparation of amine salt of Obeticholic acid in form of solvate, the amine salt of Obeticholic acid is tertiary butylamine salt of Obeticholic acid.

In some embodiment of the invention, the obtained amine salt of Obeticholic acid in the form of solvate is used during the preparation of Obeticholic acid of formula-I. In some embodiment of the invention, the tertiary butyl amine salt of Obeticholic acid in the form of solvate is used for the preparation of amorphous form of Obeticholic acid. In some other embodiment of the invention, the tertiary butyl amine salt of Obeticholic acid in form of methanol solvate is used in the preparation of amorphous form of Obeticholic acid.

Yet another aspect of the invention is to provide a process for preparation of amorphous form of Obeticholic acid comprising the step of converting tertiary butylamine salt of Obeticholic acid to amorphous form of Obeticholic acid. This embodiment also includes the purification of the amorphous form of Obeticholic acid by formation of tertiary butylamine salt. In some embodiments, the amorphous form of Obeticholic acid obtained from of tertiary butyl amine salt of Obeticholic acid is pure form having chenodeoxycholic acid impurity less than 0.5% w/w.

In some embodiment of the invention, the said process for the preparation of amorphous form of Obeticholic acid comprises the steps of:
(a) adding tertiary butylamine salt of Obeticholic acid to mixture of a water and water immiscible solvent;
(b) adjusting the pH of the biphasic mixture obtained in step (a) between 2-5;
(c) separating the organic layer from the biphasic mixture obtained in step (b)
(d) adding water to the separated organic layer obtained in step (c);
(e) adding alkali carbonate or bicarbonate solution to the biphasic mixture obtained in step (d) till the pH between 9-13;
(f) separating the aqueous layer from the biphasic mixture obtained in step (e);
(g) adjusting the pH between 3-5;
(h) optionally stirring till the solid formation; and
(i) isolating the amorphous form of Obeticholic acid.

The water immiscible solvents as in the step (a) is selected from the group comprising of esters such as ethyl acetate, n-butyl acetate and the likes; ethers such as methyl tertiary butyl ether, diethyl ether and the likes; chlorohydrocarbons such chloroform and dichloromethane; and aromatic hydrocarbons such benzene, toluene and xylene.

The alkali carbonate or bicarbonate solution as in step (e) is selected from the group including sodium carbonate, sodium bicarbonate, potassium carbonate and potassium bicarbonate.

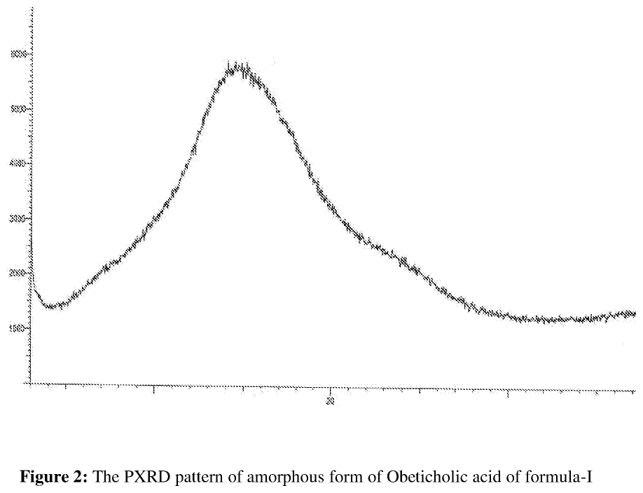

Preferably the conversion of tertiary butylamine salt of Obeticholic acid to amorphous form of Obeticholic acid is done by dissolving tertiary butylamine salt of Obeticholic acid in a suitable solvent(s); adding a water immiscible solvent preferably ethyl acetate or dichloromethane to the solution after acidifying the solution to a pH of 3-5; stirring and allowing to settle the biphasic mixture thereafter eliminating the aqueous phase; extracting the product in the organic phase with water and ammonia; adding phosphoric acid, acetic acid, dilute hydrochloric acid or citric acid slowly to the aqueous phase thus obtained and stirring for 10 to 15 hours at temperature between 25° C. to 23° C.; filtering the product and drying the product under vacuum to obtain the amorphous form of Obeticholic acid. The XRPD of the amorphous form of Obeticholic acid has been illustrated in FIG. 2.

The present invention is explained in detail with reference to the following examples described below, which are given for the purpose of illustration only and are not intended to limit the scope of the invention.

### EXAMPLES

### Example-1

### Preparation of tertiary butylamine salt of Obeticholic acid of Formula-I from methyl-3-alpha-hydroxy-6-ethylidene-7-keto-5-beta-cholanate (formula-III)

Step-A— Preparation of 3-alpha-hydroxy-6-beta-ethyl-7-keto-5-beta-cholanoic acid of formula-II: To a mixture of the compound [(E/Z)-3α-Hydroxy-6-Ethylididne-7-keto-5β-Cholan-24-oic acid methyl ester] of formula-III (45 g), 10% palladium on carbon 50% wet (4.5 g), methanol (225 ml) and water (450 ml), sodium hydroxide (5.8 g) was added and stirred for 10 minutes at 25° C. Hydrogen gas with a pressure of 4-6 kg/cm² at 25° C. was applied to the reaction mixture and stirred for 1 hour. The reaction mixture was heated to 90-100° C. and stirred for 300 minutes at the same temperature under hydrogen pressure 4-6 kg/cm². The progress of the reaction was monitored was by TLC. After completion of the reaction, the reaction mass is cooled to 25° C., filtered through hyflo. The methanol distilled from the filtrate under vacuum and then heated to 90-100° C. to obtain the compound of formula-II.

Step-B: To a mixture of the aqueous solution containing 3-alpha-hydroxy-6-beta-ethyl-7-keto-5-beta-cholanoic acid of formula-II obtained in the step-A, sodium borohydride (3.98 g), water and sodium hydroxide solution (5 mg of sodium hydroxide in 90 ml of water) was slowly added at 100° C. and stirred for 300 minutes at the same temperature. The progress of the reaction was monitored by TLC. After completion of the reaction, the reaction was cooled to 25° C. Ethyl acetate (180 ml) was added to the cooled reaction mass and stirred for 5 minutes to form a biphasic medium. The aqueous phase was separated from the biphasic mixture. The pH of the separated aqueous layer was adjusted to 2-3 using phosphoric acid, followed by the addition of ethyl acetate (450 ml) to form a biphasic mixture. The organic layer was separated from the biphasic mixture and washed with water (270 ml). Tertiary butyl amine (11.4 g) was added to the washed organic layer and stirred for 3 hours at 25° C. The resultant solid was filtered and dried to obtain compound of formula-III. Yield: 40 g.

### Example-2

### Preparation of Amorphous Obeticholic acid from tertiary-butylamine salt of Obeticholic acid

The pH of an aqueous solution of tertiary-butylamine salt of Obeticholic acid (20 g of tertiary butylamine of Obeticholic acid in 160 ml of water) was adjusted to 2 to 4 by addition of dilute hydrochloric acid solution (12 ml), followed by the addition of ethyl acetate (80 ml) to form a biphasic mixture. The organic layer was separated from the biphasic mixture and washed with water. Water (200 ml) was added to the washed organic layer to form a biphasic mixture and the pH was adjusted to 10 to 12 by the addition of ammonia. The contents were then stirred for 2 hours at 25° C. and the aqueous layer was separated. The pH of the separated aqueous layer was adjusted to 3 to 5 by the addition of phosphoric acid and stirred for 12 to 13 hours at 30° C. The resultant solid was filtered, washed with water (100 ml) and dried to obtain amorphous form of Obeticholic acid. Yield: 18 g.

### Example-3

### Preparation of Amorphous Obeticholic acid from tertiary-butylamine salt of Obeticholic acid

The pH of an aqueous solution of tertiary-butylamine salt of Obeticholic acid (20 g of tertiary butylamine of Obeticholic acid in 160 ml of water) was adjusted to 2 to 4 by addition of dilute hydrochloric acid solution (120 ml), followed by the addition of ethyl acetate (80 ml) to form a biphasic mixture. The organic layer was separated from the biphasic mixture and washed with water. Water (200 ml) was added to the washed organic layer to form a biphasic mixture and the pH was adjusted to 10 to 12 by the addition of sodium hydroxide. The contents were then stirred for 2 hours at 25° C. and the aqueous layer was separated. The pH of the separated aqueous layer was adjusted to 3 to 5 by the addition of dilute hydrochloric acid and stirred for 12 to 13 hours at 30° C. The resultant solid was filtered, washed with water (100 ml) and dried to obtain amorphous form of Obeticholic acid. Yield: 17.5 g.

### Example-4

### Preparation of tertiary butylamine salt of Obeticholic acid of Formula-I from methyl-3-alpha-hydroxy-6-ethylidene-7-keto-5-beta-cholanate (Formula-III)

Step-A: Preparation of 3-alpha-hydroxy-6-beta-ethyl-7-keto-5-beta-cholanoic acid of formula II: To a mixture of the compound [(E/Z)-3α-Hydroxy-6-Ethylididne-7-keto-5β-Cholan-24-oic acid methyl ester] of formula-III (45 g), 10% palladium on carbon 50% wet (4.5 g), methanol (225 ml) and water (450 ml), sodium hydroxide (5.8 g) was added and stirred for 10 minutes at 25° C. Hydrogen gas with a pressure of 4-6 kg/cm² at 25° C. was applied to the reaction mixture and stirred for 3 hour. The reaction mixture was heated to 90-100° C. and stirred for 15 hours at the same temperature under hydrogen pressure 4-6 kg/cm². The progress of the reaction was monitored was by HPLC. After completion of the reaction, the reaction mass is cooled to 25° C., filtered through hyflo. The filtrate was concentrated under vacuum at 60° C. to obtain a residue and cooled to 25° C. The residue was mixed with water (90 ml) and adjusted the pH of the mixture to 2-3 by the slow addition of 15% hydrochloric acid. The resultant solid was filtered, washed with water (225 ml) and dried. The dried solid was dissolved in acetone (180 ml) at 50° C., followed by the addition of water (180 ml) at 50° C. for 1 hour and stirred the contents for 1 hour at the same temperature. The stirred contents were then cooled to 25° C. and stirred for 1 hour at the same temperature. The resultant solid was filtered, washed with 45 ml of a mixture of acetone and water (1:1) and dried to obtain the titled compound. Yield: 75%.

Step-B: Preparation of Obeticholic acid: A mixture of 3-alpha-hydroxy-6-beta-ethyl-7-keto-5-beta-cholanoic acid of formula-II obtained in the step-A (100 g) and sodium hydroxide solution (11.46 g of sodium hydroxide in 1000 ml of water) was heated to 90° C. and stirred for 10 minutes, followed by the slow addition of sodium borohydride solution (prepared by mixing 22.5 g of sodium borohydride with an aqueous solution containing 0.1 g sodium hydroxide and 100 ml of water) at 90° C. for 1.5 hours. The reaction mixture was stirred for 4 hours at 90° C. The progress of the reaction was monitored was by HPLC. After completion of the reaction, the reaction was cooled to 25° C. Methyl tertiary butyl ether (700 ml) was added to the cooled reaction mass to form a biphasic mixture. The pH of the biphasic mixture was adjusted to 2 by addition of dilute hydrochloric acid solution and stirred for 45 minutes at 25° C. The aqueous phase was separated from the biphasic mixture, washed with water (200 ml) and organic layer concentrated under vacuum to obtain a residue. The residue was cooled to 35° C. mixed with dichloromethane (400 ml) and stirred to 45° C. for 45 minutes and then cooled to 25° C. The cooled reaction mass was stirred for 3 hours at 25° C. till the formation of solid. The resultant solid was filtered, washed with 100 ml of dichloromethane and dried to obtain the titled compound. Yield: 65%.

Step-C: Preparation of tertiary butylamine salt of Obeticholic acid of formula-I: To a mixture of Obeticholic acid (obtained from step-B) and acetone (930 ml), tertiary butylamine solution (25 g of tertiary butylamine in 100 ml of acetone) was added for 1 hour at 25° C. and stirred for 6 hours at the same temperature. The resultant solid was filtered, washed with 100 ml acetone and dried to obtain the titled compound.

Step-D: Purification of tertiary butylamine salt of Obeticholic acid: To a mixture of tertiary butylamine salt of Obeticholic acid (obtained from step-C) and methanol (100 ml), acetone (400 ml) was added at 50° C. and stirred the mass for 1 hour at the same temperature. The contents were then cooled to 25° C. and stirred for 1 hour at the same temperature. The resulted solid was filtered, washed with 100 ml of acetone and dried to obtain the purified tertiary butylamine salt of Obeticholic acid as a methanol solvate. Yield: 75%.

Example-5: Preparation of Amorphous Obeticholic acid from tertiary-butylamine salt of

Obeticholic acid: The pH of a biphasic mixture of tertiary-butylamine salt of Obeticholic acid (100 g), 1000 ml of water and methyl tertiary butyl ether (700 ml) was adjusted to 2 to 4 by addition of dilute hydrochloric acid solution (100 ml, 15%) and stirred for 45 minutes at 25° C. The organic layer from the biphasic mixture was separated, washed with water (200 ml) and then charcoalized with activated with carbon (5 g). Water (1000 ml) was added to the charcoalized organic layer followed by the sodium carbonate solution (50 g of sodium carbonate in 500 ml of water) to form a biphasic mixture. The biphasic mixture was maintained at the pH of 10.5 and stirred for 50 minutes at 25° C. The aqueous layer was separated from the biphasic mixture. The pH of the separated aqueous layer was adjusted to 2 by the addition of hydrochloric acid solution (15%) and stirred for 1.5 hours at 25° C. till the precipitation of solid. The precipitated solid was filtered, washed with water (1000 ml) and dried to obtain amorphous form of Obeticholic acid. Yield: 65%; Chenodeoxycholic acid Impurity (CDCA Impurity): 0.09% w/w.

Example-6: Preparation of Amorphous Obeticholic acid from tertiary-butylamine salt of obeticholic acid:

The pH of a biphasic mixture of tertiary-butylamine salt of Obeticholic acid (100 g), 1000 ml of water and methyl tertiary butyl ether (700 ml) was adjusted to 2 to 4 by addition of dilute hydrochloric acid solution (100 ml, 15%) and stirred for 45 minutes at 25° C. The organic layer from the biphasic mixture was separated, washed with water (200 ml) and then charcoalized with activated carbon (5 g). Water (1000 ml) was added to the charcoalized organic layer followed by the sodium hydroxide solution (12 g of sodium hydroxide in 120 ml of water) to form a biphasic mixture. The biphasic mixture was maintained at the pH of 10.5 and stirred for 50 minutes at 25° C. The aqueous layer was separated from the biphasic mixture. The pH of the separated aqueous layer was adjusted to 2 by the addition of hydrochloric acid solution (15%) and stirred for 1.5 hours at 25° C. till the precipitation of solid. The precipitate solid was filtered, washed with water (1000 ml) and dried under vacuum below 50° C. to obtain amorphous form of Obeticholic acid. Yield: 65%; CDCA Impurity: 0.17% w/w.

1. A process for the preparation of Obeticholic acid of formula-I having chenodeoxycholic acid impurity less than 0.5% w/w, 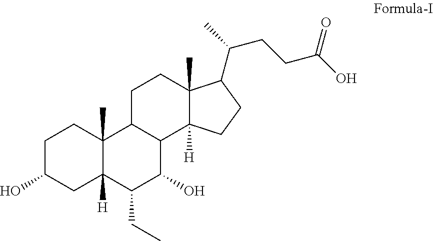 [SMILES: CC[C@H]1[C@@H](O)C2C(CC[C@]3(C)C([C@H](C)CCC(=O)O)CC[C@@H]23)[C@@]2(C)CC[C@@H](O)C[C@@H]12]
said process comprising the steps of:
(i) converting the compound of formula-III or its salt thereof to a compound of formula-II or its salt thereof 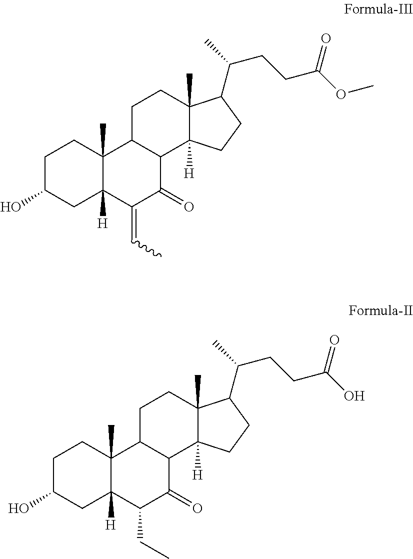
in the presence of palladium carbon in aqueous alkaline medium;
(ii) reducing the compound of formula-II or its salt thereof in the presence of sodium borohydride in an aqueous alkaline medium to obtain Obeticholic acid;
(iii) treating Obeticholic acid with tertiary butylamine to obtain a tertiary butylamine salt of Obeticholic acid; and
(iv) converting tertiary butylamine salt of Obeticholic acid to Obeticholic acid of formula-I having chenodeoxycholic acid impurity less than 0.5% w/w.

2. The process as claimed in claim 1, wherein Obeticholic acid obtained in step (iv) is the amorphous form of Obeticholic acid.

3. A solid form of tertiary butylamine salt of Obeticholic acid.

4. The solid form of tertiary butylamine salt of Obeticholic acid as claimed in claim 3, characterized by X-ray diffraction spectrum having peaks expressed as 28 values at about 9.8, 11.6, 13.8, 14.6, 16.9, 17.9 and 18.51±0.2 degrees.

5. (canceled)

6. A process for the preparation of amorphous form of Obeticholic acid comprising the step of converting tertiary butylamine salt of Obeticholic acid to amorphous form of Obeticholic acid.

7. The process as claimed in claim 6, wherein said process comprises:
(a) adding tertiary butylamine salt of Obeticholic acid to mixture of a water and water immiscible solvent;
(b) adjusting the pH of the biphasic mixture obtained in step (a) between 2-5;
(c) separating the organic layer from the biphasic mixture obtained in step (b)
(d) adding water to the separated organic layer obtained in step (c);
(e) adding alkali carbonate or bicarbonate solution to the biphasic mixture obtained in step (d) till the pH between 9-13;
(f) separating the aqueous layer from the biphasic mixture obtained in step (e);
(g) adjusting the pH between 3-5;
(h) optionally stirring till the solid formation; and
(i) isolating the amorphous form of Obeticholic acid.

8-14. (canceled)
